(19) United States
(12) Patent Application Publication
(10) Pub. No.: US 2006/0135472 A1
(43) Pub. Date: Jun. 22, 2006

(54) Use of water-soluble polymers to improve stability of diiodomethyl-para-tolylsulfone in complex matrices

(76) Inventor: Ioana Annis, Mundelein, IL (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 11/300,935

(22) Filed: Dec. 15, 2005

Related U.S. Application Data
(60) Provisional application No. 60/637,002, filed on Dec. 17, 2004.

(51) Int. Cl. A61K 31/717; A61K 31/10

(52) U.S. Cl. 514/57; 514/709

(57) ABSTRACT

The invention is a method comprising the use of diiodomethyl-para-tolylsulfone with water-soluble polymers, resulting in improved stability of diiodomethyl-para-tolylsulfone in various compositions with complex matrices. In a preferred embodiment of the invention, diiodomethyl-para-tolylsulfone is premixed with a solution of cationic polymer prior to addition to a formulation containing a complex matrix of ingredients that degrade diiodomethyl-para-tolylsulfone. As a result, diiodomethyl-para-tolylsulfone retains its chemical stability and antimicrobial activity.

### FIELD OF THE INVENTION

This invention is a method comprising the use of diiodomethyl-para-tolylsulfone (DITS, marketed by The Dow Chemical Company, Midland, Mich., under the trademark AMICAL™ antifungal agents) with water-soluble polymers, resulting in improved stability of diiodomethyl-para-tolylsulfone in various compositions with complex matrices. In a preferred embodiment of the invention, diiodomethyl-para-tolylsulfone is premixed with a solution of cationic polymer prior to addition to a formulation containing a complex matrix of ingredients that could degrade diiodomethyl-para-tolylsulfone. As a result, the diiodomethyl-para-tolylsulfone retains its chemical stability and antimicrobial activity.

### BACKGROUND

Diiodomethyl-para-tolylsulfone (DITS, marketed by The Dow Chemical Company, Midland, Mich., under the trademark AMICAL™ antifungal agent) is a biocide used for the control of microbial contamination in a variety of end-use applications. Diiodomethyl-para-tolylsulfone has extremely low solubility in water, and as a solid it has good chemical stability and compatibility with various complex formulations. DITS is a preferred material in antifungal formulations because of its excellent antimicrobial efficacy and good toxicological profile. As a solid, diiodomethyl-para-tolylsulfone has a very low reactivity. However, many aqueous compositions contain solubilizing agents and emulsifiers that increase the solubility of diiodomethyl-para-tolylsulfone and cause it to be in contact with reactive chemicals in the composition. The result is that the diiodomethyl-para-tolylsulfone may react with a broad variety of compounds and lose some or all of its antimicrobial activity.

Personal and household care formulations are typically packaged with aqueous solutions. The shelf life, or useful life of these products is set to meet consumer and retailer expectations, and includes a requirement for controlling microbial growth. Until now, DITS, an excellent fungicide with low toxicity, has not been effective for aqueous solutions with complex chemical matrices. This was due in part to the increased solubility of DITS in these matrices, resulting in higher “in-solution” concentrations of DITS, which readily reacts with other matrix components and looses its desirable antifungal activity. Soluble DITS readily reacts with a broad array of reducing and oxidizing reagents, which are used as cleaning agents in a broad variety of household and personal care items. DITS is also reactive with aldehydes and ketones, which are widely used in fragrance packages.

Water-soluble and water-dispersible polymers are frequently used as dispersing aids in aqueous formulations. In addition, some water-soluble polymers form films and coacervates, and are used in personal and household care to improve deposition of active ingredients onto hair, skin, wood, leather, and other surfaces. Cationically-modified water soluble polymers are particularly useful in improving deposition onto surfaces. However, the use of water-soluble polymers, including cationically-modified water solubility polymers, to improve the chemical stability of diiodomethyl-para-tolylsulfone has not been explored.

I have discovered that water-soluble polymers can be used to enhance the chemical stability of diiodomethyl-para-tolylsulfone in aqueous solutions with complex matrices, and therefore prolong its antimicrobial activity in products.

U.S. Pat. No. 3,726,815 describes the use of cationic polymers in a bactericide-containing detergent composition to increase the deposition of the bactericide on hair, skin or textiles. U.S. Pat. No. 4,675,178 teaches the use of a cationic polymer in a deodorant composition to improve the biocidal activity of the deodorant.

U.S. Pat. No. 4,832,950 discloses the use of several cationic polymers as dispersants for an antifungal agent (metal salt of 2-mercaptopyridine N-oxide) to generate a physically stable dispersion. The improved dispersion did not flocculate or settle after heating or freezing. The chemical stability of the antifungal agent is not discussed.

U.S. Pat. No. 5,723,112 discloses an antimicrobial hair treatment composition comprising a water soluble cationic polymer as a deposition aid.

EP 0917880 toe Wimmer, et al. describes includsion of diiodomethyl-p-tolylsulfone with cyclodextrin derivatives and the use of this inclusion complex for the preservation of products. The cyclodextrin derivatives are cyclic oligosaccarides made enzymatically from starch.

US2002068092 describes nanoparticle compositions comprising an active agent and cationic polymers adsorbed to the surface of the nanoparticle. The polymers enhance the physical stability of the formulation by minimization of settling. The chemical stability of the active agent is not discussed.

I have found that a preferred method of application is preparing a dispersion of diiodomethyl-para-tolylsulfone in a water-soluble cationic polymer, prior to adding this biocide to the remaining formulation ingredients. This method allows the diiodomethyl-para-tolylsulfone to remain chemically stable, despite the inclusion of other reactive ingredients in the formulation.

### SUMMARY OF THE INVENTION

This invention is a method of using water-soluble polymers, especially cationically-modified water-soluble polymers, together with diiodomethyl-para-tolylsulfone to increase the chemical stability of the diiodomethyl-para-tolylsulfone in a variety of aqueous formulations containing chemical matrices. In one embodiment the method comprises the pre-blending or pre-dispersion of diiodomethyl-para-tolylsulfone in such polymer solutions prior to the addition to a matrix.

### DETAILED DESCRIPTION

Diiodomethyl-para-tolylsulfone is an extremely active antifungal agent with minimum inhibitory concentration values less than about 10 ppm for a wide variety of fungi. Diiodomethyl-para-tolylsulfone is active over a wide pH range, and it has a good toxicity profile. Diiodomethyl-para-tolylsulfone also has low water solubility, less than about 10 ppm. In a broad range of applications, this low solubility enhances chemical stability, and therefore the diiodomethyl-para-tolylsulfone is very efficient.

When dissolved, or otherwise brought in contact with other chemicals, however, diiodomethyl-para-tolylsulfone is chemically reactive with a broad range of functionalities, and often resulting in compounds with little or no antifungal activity. The term “complex matrix” as used herein, includes any formulation be it liquid, gel, dispersion, solid or powder, containing more than two elements. Depending on the intended end-use, these matrices may contain surfactants, dispersing agents, fragrances, cleaning agents, such as bleach or peroxides, organic solvents, polymers, paper pulp, cement, etc. Non-limiting examples of such matrices are: household, industrial and institutional cleaning and disinfection products; medical anti-fungal formulations; industrial water including cooling water, pulp and paper and oil field; drilling muds; sump treatment for metal working systems; paints, adhesives, latex emulsions, and joint cements; building and construction materials, such as wood, wallboard, various specialty boards, including cardboard and particle board, grout, cement and insulation; cutting oils; a spray or dip treatment for textiles and leather; formulations used in the treatment of cane and beet sugar processing equipment; formulations used in the treatment of air washer or scrubber systems and in industrial fresh water supply systems; formulations used to prevent sap stain discoloration on freshly cut wood; clay and pigment slurries. Such reactions are undesirable, because they reduce the active diiodomethyl-para-tolylsulfone loading, and thus the anti-fungal resistance of the chemical matrix. While the water solubility of diiodomethyl-para-tolylsulfone is very low, its solubility is significantly increased in certain complex matrices due to the presence of organic solvents, surfactants, or salts. We have discovered a novel method of increasing the stability of diiodomethyl-para-tolylsulfone in such matrices, by delivering it together with water-soluble polymers. In a preferred embodiment of the invention, the diiodomethyl-para-tolylsulfone is pre-mixed or pre-dispersed in the polymer solution prior to addition to the formulation.

The water-soluble polymer of this invention includes any cationic, non-ionic, anionic or amphoteric polymer with a general water solubility of over 0.1%, more favored over 1%, and even more preferred readily miscible with water up to 10%. While not wishing to be bound by any theory, I hypothesize that the water-soluble polymer functions by forming a complex with the diiodomethyl-para-tolylsulfone, which decreases its chemical availability and its solubility in the matrix. Therefore, the use of polymers that comprise a hydrophobic moiety resulted in increased chemical stability of the diiodomethyl-para-tolylsulfone in the formulation.

Examples of water-soluble polymers useful for the purpose of this invention include, but are not limited to, cationic, anionic, amphoteric or non-ionic polymers, with water solubility of at least 0.1% at 25° C. Those skilled in the art will be able to choose the best-suited polymer for their needs. The compatibility of diiodomethyl-para-tolylsulfone with the polymer needs to be considered, keeping in mind the chemical reactivities of diiodomethyl-para-tolylsulfone, of the polymer, and of the potential impurities introduced in the system with the polymer.

Non-limiting examples of suitable anionic polymers are as follows: homo- and copolymers of acrylic acid; homo- and copolymers of methacrylic acid; polyacetates; anionic polyacrylamides; anionically-charged poly(amino acids), such as poly(aspartic acid) and poly(glutamic acid); polysulfonates; polysulfates; anionically-modified polysaccharides, such as cellulose acetate phthalate, cellulose acetate, and dextrin sulfates; polylactic acid.

Non-limiting examples of non-ionic polymers are as follows: poly(ethylene oxide); guar gums; cellulose and modified cellulose, such as hydroxymethyl, hydroxyethyl, and hydroxypropyl cellulose; non-ionic poly(amino acids); polyvinylpyrrolidones; polyalkylene imines. An example of preferred guar gum is Guar Gum GW 27 distributed by BJ Services Company.

Non-limiting examples of cationic polymers are as follows: cationic cellulose ethers, such as those distributed by Amerchol Corporation under the trademark UCARE™ polymers; hydrophobically-modified cationic cellulose ethers, such as those distributed by Amerchol Corporation under the trademark SOFTCAT™ polymers; cationic guar gums; hydrophobically-modified cationic guar gums; cationic polyamides; cationic poly(amino acids), such as polylysine and polyarginine; quaternized polyalkyleneimines; polyquaternary amines; and modified polypyridines. In a preferred embodiment of this invention, the polymer is selected from the classes of cationic cellulose ethers, hydrophobically-modified cationic cellulose ethers, or cationic guar gums. In a most preferred embodiment of this invention the polymer is a hydrophobically-modified cationic cellulose ether.

The experimental examples provided herein describe the stabilization of diiodomethyl-para-tolylsulfone in a shampoo matrix. Shampoo matrices have high surfactant concentration that significantly increase the solubility of diiodomethyl-para-tolylsulfone. In addition shampoos may contain additives to enhance shine and manageability, control dandruff and other scalp conditions, protect hair from sunlight, and other functions. The variety and complexity of additives used in shampoo matrices may affect its stability. Indeed, the stability of diiodomethyl-para-tolylsulfone in the control samples formulated without polymer is low (˜60% decomposition after 8 weeks at 40° C.). I have discovered that the use of polymers in conjunction with diiodomethyl-para-tolylsulfone increases the stability of the biocide. The use of diiodomethyl-para-tolylsulfone pre-mixed with the polymer solution further increases the stability of diiodomethyl-para-tolylsulfone. In certain cases, I have seen no decomposition after exposure to 40° C. for 8 weeks.

While the stabilization of diiodomethyl-para-tolylsulfone in shampoo was chosen for proof of concept, this invention is not limited to shampoo applications. Similar benefits could be gained in various other applications where the chemical stabilization of diiodomethyl-para-tolylsulfone is desired. Non-limiting examples of such applications include: all personal care applications wherein antifungal activity is desired; household, industrial and institutional cleaning and disinfection; medical anti-fungal applications; industrial water treatment including cooling water, pulp and paper and oil field treatment; drilling mud preservative; sump treatment for metal working systems; in-can and dry-film preservation of paints, adhesives, latex emulsions, and joint cements; building and construction material preservation, such as wood, wallboard, various specialty boards, including cardboard and particle board, grout, cement and insulation; preservation of cutting oils; a spray or dip treatment for textiles and leather; as a component of anti-fouling paints; protecting paint films; protecting cane and beet sugar processing equipment; treatment of air washer or scrubber systems and in industrial fresh water supply systems; preventing sap stain discoloration on freshly cut wood; treatment of clay and pigment slurries. This invention is particularly useful for aqueous solutions of DITS that are stored without adding more biocide during storage.

Different polymers will be optimal depending on the product formulation; those skilled in the art would identify the best candidates for their specific application. In a preferred embodiment of this invention, the stabilizing polymer is a cationic polymer. In various applications, cationic polymers are used to impart certain desirable properties to the final formulation such as retention of actives, deposition of actives on skin or hair, improved conditioning and manageability of hair, etc. The approach introduced herein, whereby diiodomethyl-para-tolylsulfone is premixed or pre-dispersed in a polymer solution before addition to the final formulation has the additional benefit of improving the retention or deposition of the DITS on the treated material, be it paper, wood, hair, skin, etc.

The weight ratio of polymer to DITS ratio can be adjusted, depending on the particular polymer and application. One skilled in the art can optimize the ratios for a particular product performance. In general, the weight ratio of polymer to diiodomethyl-para-tolylsulfone is (10⁻⁴ to 10⁴): 1; more preferably (10⁻² to 10²):1; and more preferaby as (0.01 to 0.1):1.

### EXAMPLES

The approach was tested on generic formulations prepared as described herein. Raw materials used were as follows: Standapol* EA-3 (alkyl ether sulfates) and Standapol* A (ammonium lauryl sulfate) from Cognis Corporation (Cincinnati, Ohio); Sodium lauroamphoacetate (LAA, Mackam HPL-28) from McIntyre Group (University Park, Ill.); GW 27, a proprietary blend of guar gum, aliphatic carboxylic acid and alkaline metal oxides, from BJ Services Company (Houston, Tex.); Celvol* 205S, polyvinyl alcohol, from Celanese (Dallas, Tex.); Glydant* (DMDM hydantoin) from Lonza Corporation (Fairlawn, N.J.); Floral for antidandruff shampoo fragrance oil 04f/4835 from Fragrance Resources (Clifton, N.J.); Jaguar* C13S, guar hydroxypropyltrimonium chloride, Jaguar* C162, hydroxypropylguar hydroxypropyltrimonium chloride, and Jaguar* Excel, guar hydroxypropyltrimonium chloride from Rhodia (Cranbury, N.J.); all other cationic cellulose ether polymers were obtained as samples from Amerchol Corporation (Piscataway, N.J.); all other chemicals were obtained from Aldrich (Milwaukee, Wis.). AMICAL™ 48 (diiodomethyl-para-tolylsulfone) was air milled to a D₅₀ of 2 microns prior to use.

All analytical data were obtained by high performance liquid chromatography (“HPLC”) analysis for content for diiodomethyl-p-tolylsulfone (DITS) and monoiodomethyl-p-tolylsulfone (MITS). Analysis was done on an Agilent HP system using a Phenomenex Bondclone 10 micron C18 reverse phase column (300×3.9 mm, 10 microns) at a flow rate of 2 mL/min, isocratic 55% acetonitrile in water for 17 min. Under these conditions DITS elutes at 3.9-4.3 min and MITS elutes at 2.9-3.3 min, depending on the shampoo matrix.

### Example 1

### Stability of DITS in Generic Shampoo Formulation, When Formulated as a Premix with Polymer. Method 1.

All percentages are by weight, based on the total formulation. This procedure was followed for the following polymers: SOFTCAT™ SL100 at 0.25 wt. % concentration; SOFTCAT™ SL400 at 0.25 wt. % concentration; and Jaguar* C13S, at 0.25 wt. % concentration. In all cases, AMICAL™ 48 milled to a D₅₀=2 microns was used (diiodomethyl-p-tolylsulfone content 92.5%). Detailed here is the example involving SOFTCAT™ SL 400, at 0.25% concentration.

To a 250 mL glass jar were added 24.81 g water and 0.2526 g SL 400. The mixture was stirred vigorously for 1.5 h. Then, 1.0302 g milled AMICAL™ diiodomethyl-para-tolylsulfone was added, and the mixture was stirred for an additional 1.5 hours. After that, the premix was heated to 68° C. in a hot water bath, and a pre-blended mixture of Standapol* EA-3 (32.98 g), Standapol* A (11.16 g), and LAA (16.70 g) was added with agitation. 12.9 g of DI water was added next, and stirring was continued in the hot water bath. After 10 min, mL of a 10% aqueous citric acid solution was added to adjust the pH. After an additional 10 min, the temperature of the mixture was recorded (68° C.) and the heating was stopped. The mixture was stirred on high speed for another hour, until it cooled. At that point, 0.31 g Glydant* and 0.30 g fragrance were added, and the sample was adjusted with water to 100 g formulation. Stirring continued for another hour. HPLC analysis showed 0.8% DITS content at time zero. After storage for 8 weeks at 40° C., HPLC analysis revealed 0.79% DITS content, accounting for all the added DITS within the limit of error for the analytical technique.

### Example 2

### Stability of DITS in Generic Shampoo Formulation, When formulated as a Premix with Polymer. Method 2.

This procedure was followed for the following polymers: SOFTCAT™ SL 400, at 0.5% concentration; guar gum GW 27, at 0.25% concentration; Jaguar* C13S at 0.25% concentration; POLYOX™ N-3000 polymers at 0.5% concentration; Celvol* 205S at 0.25% concentration; UCARE™ JR 400 at 1% concentration; poly(acrylic acid) (35% soln. in water, MW 250,000) at 5% concentration. In all cases, AMICAL™ 48 (diiodomethyl-para-tolylsulfone) milled to a D₅₀=2 microns was used (diiodomethyl-p-tolylsulfone content 92.5%). Detailed here is the example involving SOFTCAT™ SL 400, at 0.5% initial concentration.

In a 10 mL glass vial were combined 0.5064 g SOFTCAT™ SL 400 and 1.0187 g milled diiodomethyl-para-tolylsulfone. The vial was capped and rolled in-hand for 2 min, after which the powder blend was added with agitation to a 250 mL glass jar containing 24.54 g water. The mixture was stirred vigorously for 1.5 h using an overhead stirrer. After that, the diiodomethyl-para-tolylsulfone-polymer premix was heated to 45° C. in a hot water bath, and a pre-blended mixture of Standapol* EA-3 (33.19 g), Standapol* A (10.72 g), and LAA (17.06 g) was added with agitation. 13.12 g of DI water was added next, and stirring was continued in the hot water bath. After 10 min, mL of a 10% aqueous citric acid solution was added to adjust the pH. After an additional 10 min, the temperature of the mixture was recorded (57° C.) and the heating was stopped. The mixture was stirred on high speed for another hour, until it cooled. At that point, 0.37 g Glydant* and 0.3 g fragrance were added and the sample was adjusted with water to 100 g formulation. Stirring continued for another hour. HPLC analysis showed 0.77% DITS content at time zero. After storage for 8 weeks at 40° C., HPLC analysis revealed 0.66% DITS content, accounting for 85.7% of the initially added DITS.

### Example 3

### Stability of DITS in Generic Shampoo Formulation, When Added to the Surfactant Blend

This procedure was followed for all the polymers listed in Table 1, at the stated polymer concentration. In all cases, AMICAL™ 48 milled to a D₅₀=2 microns was used (diiodomethyl-p-tolylsulfone content 92.5%). Detailed here is the example involving SOFTCAT™ SL 400 at 0.25% concentration.

To a 250 mL glass jar were added 24.82 g water and 0.2644 g SOFTCAT™ SL 400. The mixture was stirred vigorously for 1.5 h. Separately, in a 250 mL beaker were combined Standapol* EA-3 (33.57 g), Standapol* A (11.08 g), and LAA (16.13 g). The mixture was stirred vigorously using an overhead stirrer, and after 10 min, 1.0120 g milled DITS (AMICAL™ 48) was added, followed by 5.0 g water. The DITS-surfactant blend was stirred for 1.25 hours. After that, the DITS—surfactant blend was added with agitation to the polymer solution which had been heated to 58° C. in a hot water bath. 8.12 g of deionized water was added next, and stirring was continued in the hot water bath. After 10 min, 1 mL of a 10% aqueous citric acid solution was added to adjust the pH. After an additional 15 min, the temperature of the mixture was recorded (58° C.) and the heating was stopped. The mixture was stirred on high speed for another hour, until it cooled. At that point, 0.44 g Glydant* and 0.3 g fragrance were added, and the sample was adjusted with water to 100 g formulation. Stirring continued for another hour. HPLC analysis showed 0.79% DITS content at time zero. After storage for 8 weeks at 40° C., HPLC analysis revealed 0.71% DITS content, accounting for 89.9% of the DITS initially added to the formulation.

### Example 4

### Stability of DITS in the Absence of Polymer. Control Experiment

DITS (AMICAL™ 48) milled to a D₅₀=2 microns was used (diiodomethyl-p-tolylsulfone content 92.5%).

To a 250 mL glass jar were added Standapol* EA-3 (33.32 g), Standapol* A (10.72 g), and LAA (16.78 g). The mixture was stirred vigorously for 10 minutes, using an overhead stirrer. After 10 min, 0.7521 g milled diiodomethyl-para-tolylsulfone (AMICAL™ 48) were added, followed by 6.0 g water. The DITS-surfactant blend was stirred for 1.0 hour. After that, 1 mL of a 10% aqueous citric acid solution was added to adjust the pH, and the stirring continued for another hour. At that point, 0.41 g Glydant* and 0.31 g fragrance were added, and the sample was adjusted with water to 75 g formulation. Stirring continued for another hour. HPLC analysis showed 1.02% DITS content at time zero. After storage for 8 week at 40° C., HPLC analysis revealed 0.44% DITS content, accounting for 43.2% of the DITS initially added to the formulation.

### Example 5

### Diiodomethyl-Para-Tolylsulfone Deposition on In-Vitro Skin. Deposition Efficiency of Various Formulations

In-vitro skin was purchased from IMS Testing Group. The skin was cut in rectangles of 3.2×5.1 cm, and hydrated overnight in a chamber containing glycerin-water, 1:5.8. During the treatment with shampoo, the skin piece was placed on a firm sponge wrapped in plastic to simulate the resistance of the skin. 0.3 g of formulation was deposited on the skin (small dots covering the surface) and the shampoo was rubbed over the skin with an ungloved finger for one minute. Immediately after, the skin was rinsed for one minute with water (28-34° C.), using a spray bottle held 15 cm away from the skin. For consistency, all pieces of skin were sprayed at the same setting of the spray bottle. After rinsing, the skin was dried overnight in air, cut in small pieces, and extracted with acetonitrile for HPLC quantification of DITS content.

Three formulations were tested in triplicate: a control in which diiodomethyl-para-tolylsulfone was added to the surfactant blend and no polymer was present (prepared as in Example 4), a formulation in which diiodomethyl-para-tolylsulfone was added as a pre-dispersion in a solution of SOFTCAT™ SL 400 0.5% concentration (prepared as in Example 2), and a formulation containing 0.5% SOFTCAT™ SL 400 in which diiodomethyl-para-tolylsulfone was added to the surfactant blend (prepared as in Example 3). HPLC analysis showed that: 0.003 μg DITS/cm² skin were deposited using the control formulation with no polymer; 0.166 μg DITS/cm² skin were deposited using formulation prepared as in Example 2; 0.01 μg DITS/cm² skin were deposited using formulation prepared as in Example 3.

TABLE 1
Stability of DITS in a generic shampoo matrix, after exposure to 40° C. for 8
weeks.

| | Initial | % DITS left | % DITS left | % DITS left |
|---|---|---|---|---|
| | polymer | when added | when added | when added to |
| | concentration^{a} | as premix. | as premix. | surfactant |
| Polymer used | (%) | Met. 1^{b} | Met. 2^{c} | blend^{d} |
| None (Control - Ex. 4) | — | — | — | 43.2 |
| SOFTCAT ™ SL 100 | 0.25 | 81.5 | — | 97.9 |
| SOFTCAT ™ SL 400 | 0.25 | 98.8 | — | 89.9 |
| SOFTCAT ™ SL 400 | 0.5 | — | 85.7 | 65.5 |
| POLYOX ™ N-3000 | 0.5 | — | 77.2 | 90 |
| Jaguar* C13S | 0.25 | 84.4 | 71.4 | 87.5 |
| GW 27 guar gum | 0.25 | — | 75.6 | 67.4 |
| UCARE ™ JR 400 | 1.0 | | 70.0 | 62.8 |
| Celvol* 205 S | 0.25 | | 76.3 | 75 |
| Poly(acrylic acid) | 5.0 | — | 96.5 | 96.9 |

^{a}Concentration of the polymer in the final formulation.
^{b}Addition of diiodomethyl-para-tolylsulfone as described in Example 1.
^{c}Addition of diiodomethyl-para-tolylsulfone as described in Example 2.
^{d}Addition of diiodomethyl-para-tolylsulfone as described in Example 3.
™A Trademark of The Dow Chemical Company (“Dow”) or an affiliated company of Dow.
*Standapol is a trademark of Cognis Corporation, Glydant is a trademark of Lonza Corporation, Jaguar is a trademark of Rhodia, and Celvol is a trademark of Celanese.

What is claimed is:

1. An aqueous complex matrix formulation comprising didiodmethyl-para-tolylsulfone, and a water-soluble polymer.

2. The aqueous formulation of claim 1 further comprising surfactants selected from anionic, cationic, non-ionic or amphoteric surfactants.

3. The aqueous formulation of claim 1 where the water-soluble polymer is a cationic polymer.

4. The aqueous formulation of claim 1 where the cationic polymer is a cationic cellulose ether.

5. The aqueous formulation of claim 4 wherein the cationic cellulose ether comprises hydrophobic substitution.

6. The aqueous formulation of claim 4 where the polymer is a hydroxyethylcellulose polymer with cationic substitution of trimethylammonium and dimethyldodecylammonium.

7. The aqueous formulation of claim 1, where the polymer is a nonionic cellulose polymer.

8. The aqueous formulation of claim 1 where the ratio of polymer to diiodomethyl-para-tolylsulfone is in a range between 10⁻⁴ to 10⁴ parts by weight polymer to one part diiodomethyl-para-tolylsulfone.

9. The aqueous formulation of claim 1 where the ratio of polymer to diiodomethyl-para-tolylsulfone is in a range between 10⁻² to 10² parts by weight polymer to one part diiodomethyl-para-tolylsulfone.

10. The aqueous formulation of claim 1 where the ratio of polymer to diiodomethyl-para-tolylsulfone is in a range between 0.01 to 0:1 parts by weight polymer to one part diiodomethyl-para-tolylsulfone.

11. A method of increasing the chemical stability of diiodomethyl-para-tolylsulfone in a complex matrix comprising addition of a sufficient amount of a water-soluble polymer to the matrix.

12. The method of claim 11 further comprising adding the complex matrix to a shampoo.

13. The method of claim 11 further comprising adding the complex matrix to a household cleaning solution.

14. The method of claim 11 further comprising adding the complex matrix to a paint.

15. The method of claim 11 further comprising adding the complex to building materials.

16. A method of addition of diiodomethyl-para-tolylsulfone to a complex matrix comprising pre-mixing the diiodomethyl-para-tolylsulfone in a water-soluble polymer followed by adding the pre-mixture to the complex matrix.

17. The method of claim 16 further comprising adding the complex matrix to a shampoo.

18. The method of claim 16 further comprising adding the complex matrix to a household cleaning solution.

19. The method of claim 16 further comprising adding the complex matrix to a paint.

20. The method of claim 16 further comprising adding the complex to building materials.
